(19) United States
(12) Patent Application Publication
(10) Pub. No.: US 2011/0251121 A1
(43) Pub. Date: Oct. 13, 2011

(54) USE OF CYSTEINE-RICH WHEY DERIVED PROTEIN IN PATIENTS RECEIVING CHEMOTHERAPY AND/OR RADIOTHERAPY TO IMPROVE PATIENT SURVIVAL

(75) Inventors: Wulf DROGE, Vaudreuil (CA); Gustavo BOUNOUS, Vaudreuil (CA); John H. MOLSON, Vaudreuil (CA)

(73) Assignee: 2458781 CANADA INC., Vaudreuil-Dorion (CA)

(21) Appl. No.: 13/165,002

(22) Filed: Jun. 21, 2011

Related U.S. Application Data
(62) Division of application No. 12/210,030, filed on Sep. 12, 2008, now abandoned.

(51) Int. Cl. A61K 38/16; A61P 25/24; A61P 25/22; A61P 3/02

(52) U.S. Cl. 514/4.8; 514/5.6

(57) ABSTRACT

A cysteine-rich undenatured whey-derived protein formulation did not interfere with the tumor-cytotoxic effects of chemotherapy and radiation therapy and did not have a negative effect on the clinical outcome, that is, negatively affect survival and increase mortality. Indeed, use of a high-cysteine undenatured whey derived protein in the treatment of cancer patients resulted in an increase in patient survival.

### BACKGROUND OF THE INVENTION

In cancer patients, oxidative stress is both a curse and a blessing. Oxidative stress plays a major role in the tumor-toxic effects of chemotherapy and/or radiotherapy and, incidentally, also has substantial adverse effects on the host tissue of the patients. Evidence indicates that loss of body cell mass (cancer cachexia) results to some extent from aberrant inflammation (13, 15) and is significantly correlated with an oxidative shift in plasma redox status, as indicated by a decreased ratio of reduced to oxidized cysteine (16). Substantial weight loss usually compromises the quality of life of cancer patients and is a contributor to morbidity and mortality (5, 10, 18, 25). Attempts to prevent the loss of body cell mass by nutritional intervention have, by and large, not been satisfactory (12, 13, 15, 22, 25, 27). A preliminary study of patients with different types of cancer revealed, however, that treatment with the glutathione (GSH) precursor N-acetylcysteine reversed the loss of body cell mass and the oxidative shift in plasma redox status (16). However, this study did not report to a survival benefit. Several redox-regulated signaling pathways are known to be involved in catabolic processes (reviewed in 11).

Biologically active undenatured whey protein concentrates and isolates have been previously disclosed by Gustavo Bounous et al, which are effective for enhancement of tissue GSH levels and effective on the immune response, host resistance to and treatment of cancer, and immuno enhancement: U.S. Pat. Nos. 5,230,902; 5,290,571; 5,451,412; 5,456,924; and 5,888,552; as well as European Patent 0374390 and 0375852, all of which are incorporated herein by reference.

In particular, Bounous et al had previously disclosed anti-cancer therapeutic compositions containing whey protein concentrate, wherein increasing cellular GSH protected target cells against the effects of a carcinogen (U.S. Pat. No. 5,888,552). As well, in particular, the whey protein concentrate was shown useful in methods for treating cancer. Without being bound to any specific theory, the inventors believed that the increased availability of substrate for GSH synthesis, due to undenatured whey protein delivering glutamyl cysteine, a potent cysteine delivery system, is involved in inhibition of replication of formed cancer cells.

However, as the tumor-cytotoxic effects of cancer chemotherapy and radiotherapy typically involve oxidative stress, concern has been expressed that antioxidative treatment may interfere with these therapies and thus exacerbate mortality (see 9, 4, 24). As cysteine is a limiting biosynthetic precursor of glutathione (GSH), it is expected to ameliorate the oxidative stress.

Treatment of cancer with radiation and/or chemotherapeutic agents generates free radicals in cells and such therapies rely on their oxidative damage to eradicate the cancerous cells. Thus, there is concern and uncertainty whether antioxidants that protect normal cells from acute and long term free radical damage may afford the same protection to tumor cells and hinder the overall outcome of cancer therapy and indeed exacerbate mortality, that is, reduce patient survival. (4).

### SUMMARY OF THE INVENTION

The present invention relates to a cysteine-rich protein formulation and its use in the treatment of cancer patients to improve patient survival and quality of life.

Through diligent study, the inventors herein have surprisingly discovered a high cysteine-rich undenatured whey derived protein which, when used with cancer patients treated by chemotherapy and/or radiation therapy, exhibited an increase in patient survival in addition to improvements in other indicia such as reversal of cancer-related weight loss, reversal of loss of body cell mass, improvements in hand grip force and quality of life, Karnofsky status, feeling of anxiety, nervousness and worry, appetite and depression.

As such, in view of the aforesaid conflicting aspects, the inventors have determined that a cysteine-rich undenatured whey-derived protein formulation did not interfere with the tumor-cytotoxic effects of chemotherapy and radiation therapy and this did not have a negative effect on the clinical outcome, that is, negatively affect survival and increase mortality, as feared by some. Indeed, the inventors have discovered that use of a high-cysteine undenatured whey derived protein in the treatment of cancer patients resulted in an increase in patient survival.

Accordingly, in one aspect, the present invention is directed to a cysteine-rich undenatured whey derived protein and formulations thereof for use in treatment of cancer patients, to improve patient survival.

In another aspect, the invention relates to a dosage of a cysteine-rich whey derived protein in, a range about 5 to 30 grams of protein per day, preferably in a range of about 5-25 g/day, more preferably about 13-20 g/day and most preferably about 13 g/day.

In another aspect, the present invention relates to the use of a cysteine-rich whey derived protein in the treatment of cancer patients and methods of treatment using the said protein, to improve patient survival, along with other indicia of quality of life, particularly in use with patients undergoing chemotherapy, radiation therapy or both; and the use of said cysteine-rich protein to prepared formulations or medicaments for such use.

### BRIEF DESCRIPTION OF THE FIGURES

FIG. 1: Trial profile of the casein (CAS) group and cysteine-rich protein (CysP) group (IMN 1027).

FIG. 2: Kaplan-Meier survival curves of the lung cancer patients. Solid lines: Casein group; Dashed lines: cysteine-rich protein Group (a) Survival curves of all 66 lung cancer patients recruited into the study; (b) Group of 35 evaluable lung cancer patients (p=0.024); (c) Group of 28 evaluable lung cancer patients with chemotherapy or radiotherapy or both (n=0.058).

### DETAILED DESCRIPTION OF THE INVENTION

The inventors have determined that a cysteine-rich undenatured whey derived protein and formulations thereof improves patient survival for patients undergoing chemotherapy and radiation therapy. In a placebo-controlled double-blind clinical trial, casein was replaced with a high-cysteine whey-derived protein formulation. Specifically, the present inventors investigated if patient survival may be ameliorated in cancer patients, along with other quality of life indicia, such as body weight loss and body cell mass, by the high-cysteine whey-derived protein formulation compared with casein. Casein (i.e., the protein base of the majority of clinically used enteral nutritional supplements) was used as it contains only minute quantities of cysteine. As cysteine is a limiting biosynthetic precursor of GSH, it is expected to ameliorate the oxidative stress. The high-cysteine whey derived protein was designed to have an even higher cysteine content than normal whey protein (see Table 1).

TABLE 1
Amino acid composition* of the cysteine-rich protein
(CysP) and the control protein (casein).

| | CysP | CASEIN |
|---|---|---|
| cys | 0.50 | 0.05 |
| met | 0.14 | 0.18 |
| glu | 1.15 | 1.41 |
| asp | 1.04 | 0.56 |
| leu | 1.02 | 0.66 |
| ile | 0.46 | 0.37 |
| val | 0.46 | 0.49 |
| ala | 0.55 | 0.32 |
| ser | 0.39 | 0.50 |
| gly | 0.22 | 0.20 |
| lys | 0.74 | 0.49 |
| arg | 0.18 | 0.26 |
| his | 0.11 | 0.15 |
| pro | 0.35 | 0.88 |
| phe | 0.23 | 0.28 |
| tyr | 0.24 | 0.25 |
| thr | 0.40 | 0.32 |
| trp | 0.08 | 0.03 |

*Amino acids are indicated as moles/kg.

Both proteins are approximately isocaloric and isonitrogenous. A comparison of these two protein supplements has to be seen in the context of the general protein intake. A few results point to the generally low protein intake of patients with advanced cancer, even in patients who consumed commercially available nutritional supplements (14, 17). According to a recent review on protein requirements (20), this level of protein intake would likely be insufficient to support nitrogen balance, even in healthy individuals of a similar age. In several recent studies of pancreatic cancer patients, complex protein-containing supplements have mediated modest improvements, but the specific contribution of the protein remained unclear (1, 2, 14). In view of the notoriously low protein intake of cancer patients, it is believed that the quality of the protein is particularly important because any deficiency in the composition of a given protein may not be overcome by increasing the protein intake.

### EXAMPLES

### I. Patient Enrollment, Trial Profile, Baseline Characteristics, and Compliance

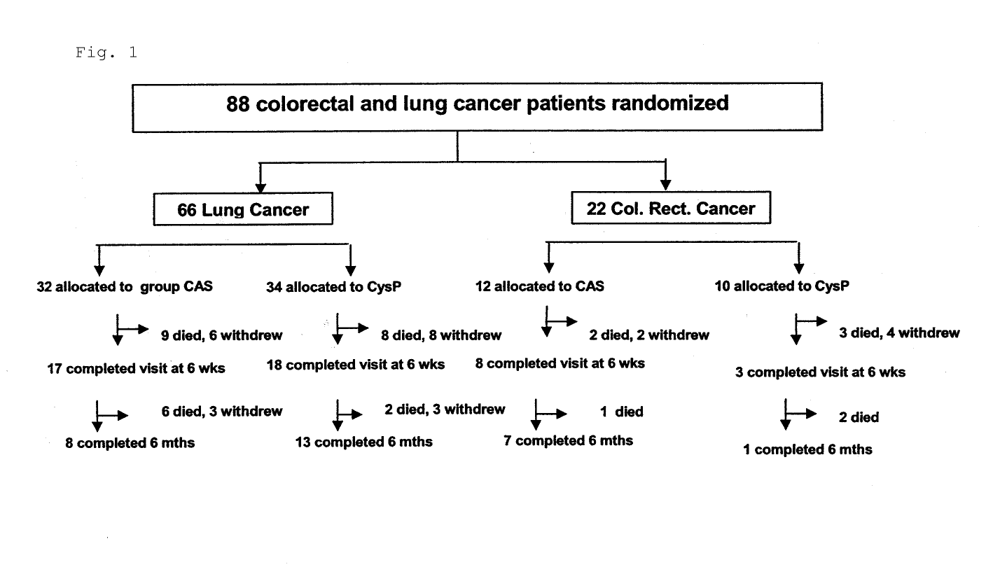

Patients were enrolled between October 2003 and February 2006 (Appendices 1-5). Sixty-six patients with lung cancer and 22 with colorectal cancer were recruited and randomly assigned to the treatment groups in two separate strata. Only seven patients consumed a minimum of 75% of their study medication (see Appendix 2) as required for the “per protocol” (PP) analysis according to the trial protocol. Therefore included in the analysis were all patients who returned their canisters and completed at least the second visit (the “evaluable patients”). The trial profile (FIG. 1) and the baseline data of the evaluable colorectal cancer patients revealed that they were not well matched, and their numbers too small to be statistically meaningful. [Specifically, the mean age (63.6:±:10.1 vs. 41.7:±7.0 years), baseline TNF-α levels (1.8±0.7 vs. 3.1±1.9 pg/ml), plasma glutamine levels (592±129 vs. 461±100 μM), and ESAS 8 (1.25±2.82 vs. 7.00±1.73) were significantly mismatched baseline parameters). Therefore, only the results from the stratum of lung cancer patients (Table 2) are reported. The mean compliance of the evaluable lung cancer patients with casein or the cysteine-rich protein was 44±34% and 42±29%, respectively, implying that the patients consumed, on the average, about 13 g/day of either protein. The reference to protein is pure protein, without any additives.

TABLE 2
Baseline characteristics*

| Assignment: - | Casein | CysP |
|---|---|---|
| N | 17 | 18 |
| | (32) | (34) |
| Female/Male | 6/11 | 4/14 |
| | (11/12) | (6/28) |
| Age (y) | 63.5 ± 10.9 | 64.3 ± 10.7 |
| | (63.6 ± 11.4) | (63.8 ± 10.1) |
| Height(cm) | 167.2 ± 8.4 | 169.5 ± 11.7 |
| Body weight (kg) | 64.5 ± 16.7 | 66.1 ± 14.3 |
| Body cell mass (kg) | 22.5 ± 6.8 | 22.4 ± 4.6 |
| CRP (mg/L) | 47.4 ± 45.6^{†,††} | 35.3 ± 54.1^{†,††} |
| | (73.3 ± 82.7)^{‡‡} | (60.1 ± 81.3)^{‡‡} |
| TNFα (pg/mL) | 15.5 ± 52.3^{‡,∥∥} | 2.6 ± 1.7^{‡,∥∥} |
| | (10.4 ± 38.5)*** | (2.6 ± 1.5)*** |
| IL-6 (pg/mL) | 6.2 ± 3.9 | 4.6 ± 3.3 |
| glutamine (μM) | 551 ± 160^{§} | 564 ± 147 |
| | (513 ± 182) | (566 ± 144) |
| cystine (μM) | 29.0 ± 15.5^{§} | 30.7 ± 14.9 |
| RBC-GSH (μM) | 476 ± 219^{§} | 443 ± 292 |
| Hand Grip Force (kg) | 28.6 ± 10.7 | 29.0 ± 8.6 |
| Karnofsky index (units) | 78.2 ± 7.3 | 78.9 ± 8.3 |
| ESAS 8 (units) | 2.94 ± 3.55^{§} | 1.67 ± 2.59 |
| Constipation | (2.59 ± 3.23) | (2.09 ± 2.84) |

*Data ± S.D. refer to the patients who completed at least two visits. Data in brackets refer to the total group of randomized patients. All groups consisted mainly of Caucasians (>85%).
^{†}C-reactive protein (CRP) data were only available from 15 casein and 16 IMN1207 (CysP) treated patients.
^{‡}TNFα data were available from 15 casein and 17 IMN1207 (CysP) treated patients.
^{§}Data were available from 16 casein treated patients.

The meaning of whey protein and sources for same will be that as would be understood by a skilled person and reference may be had to the previously referenced patents of Bounous et al for this purpose, as well as for standard techniques to produce undenatured whey protein, isolates and concentrates. For example, whey proteins, protein factions, and peptides are typically isolated and purified either by ultra filtration or by ion exchange chromatography, according to widely used standard procedures. The cysteine-rich protein of the herein application may be obtained through the standard techniques that will be known to a skilled person, of producing whey proteins, undenatured, e.g. Glanbia, Davisco. Whey protein, isolate, as well as peptides and protein fractions, with different cysteine contents may be mixed in such a way to obtain a cysteine content described in the present application. Once obtained, formulations of whey protein concentrates, isolates, peptides, protein fractions and mixtures thereof may be prepared into suitable formulations in the manner well known to a skilled person and again reference may be had to the previously referenced patents of Bounous et al.

Patient cancer treatment was by radiation therapy and/or chemotherapy. Such therapies would be understood by a skilled person, (see for example, reference (4)). For the present study, Chemotherapeutic drugs used either alone or in combination were: Carboplatin, Cisplatin, Oxaliplatin, Gemcitabine, Capecitabine, Taxotere, Etoposide, Leucovorine, Irinotecan, 5-Fluorouracil, and Cyclophosphamide.

### II. Positive: Effect of the Cysteine-Rich Protein on the Survival of Patients with Chemotherapy and/or Radiotherapy

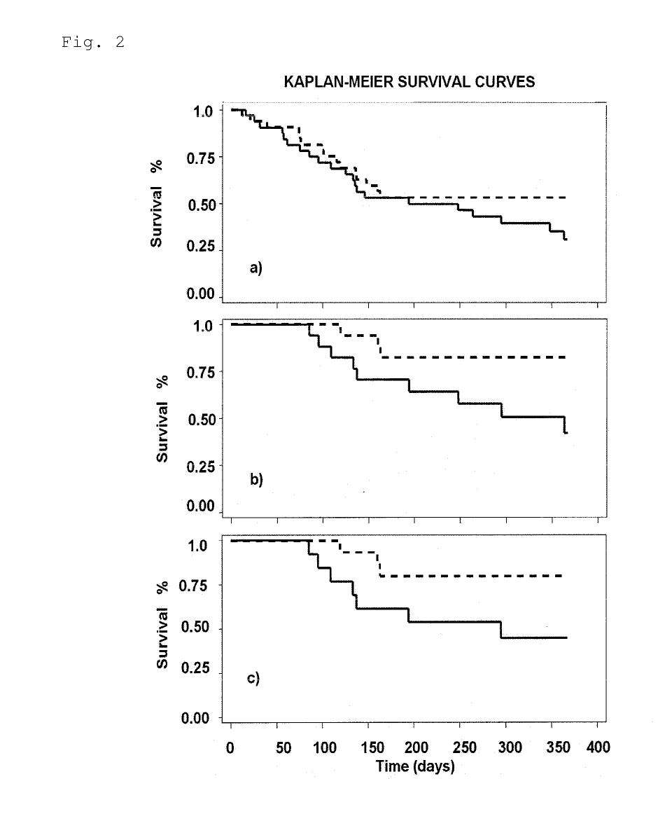

Of the 66 randomized lung cancer patients and 22 colorectal cancer patients, 25 and 36 patients with lung cancer and 8 and 15 patients with colorectal cancer died during the trial of disease-related causes within 6 months and 12 months, respectively (see FIG. 1). The Kaplan-Meier survival curves (see Appendix 8) of the two treatment arms of the totalling cancer group were not different from one another (FIG. 2a), whereas the two treatment arms of the 35 evaluable patients (FIG. 2b) were different (p=0.024; 95% hazard ratio, 0.067 to 0.916). Approximately 80% of the patients treated with cysteine-rich protein and <50% of the patients in the control group survived 12 months. The survival of lung cancer patients with chemotherapy and/or radiotherapy (n=28) (FIG. 2c) also showed a positive trend in the group with the cysteine-rich protein (p=0.058).

Patients completing the 6-month visit on the cysteine-rich protein showed, in contrast to the casein-treated group, a significant increase in body cell mass and hand-grip force (see Appendix 7) and a trend (p=0.09) in the Karnofsky status compared with baseline values (Table 3). The treatment arms, however, were not significantly different in Kornofsky status, perhaps because of the small number of surviving patients.

TABLE 3
Effect of protein supplements on changes in objective parameters

| | | | | | Mean of 6 wks, 3 mths, | |
|---|---|---|---|---|---|---|
| | 6 mths values* | | Last observation (LOCF)^{†} | | and 6 mths values^{‡} | |
| rel. or abs. | Casein | CysP | Casein | CysP | Casein | CysP |
| changes | n = 8 | n = 13 | n = 17 | n = 18 | n = 17 | n = 18 |
| Body weight | −1.01 ± 8.52 | +2.38 ± 7.21 | −2.63 ± 8.07^{§} | +2.50 ± 6.74^{§} | −1.88 ± 5.46^{\|\|} | +1.79 ± 4.45^{\|\|} |
| (%) | CI: (−8.14/+6.11) | (−1.98/+6.73) | (−6.78/+1.52) | (−0.85/+5.85) | (−4.69/+0.93) | (−0.43/+4.00) |
| Body cell mass | +10.34 ± 14.10 | +14.09 ± 15.84** | −5.47 ± 34.63^{††} | +11.55 ± 18.05^{††,‡‡} | −2.85 ± 30.93^{§§} | +8.34 ± 17.89^{§§} |
| (%) | CI: (−23.00/+43.69) | (+3.46/+24.73) | (−23.27/+12.33) | (+1.93/+21.16) | (−18.76/+13.05) | (−1.20/+17.87) |
| Hand grip | +8.49 ± 16.21 | +12.41 ± 16.52^{\|\| \|\|} | −2.22 ± 22.57 | +2.57 ± 24.75 | +0.17 ± 18.38 | +2.50 ± 18.87 |
| force (%) | CI: (−6.49/+23.48) | (+2.43/+22.39 | (−13.83/+9.38) | −(9.73/+14.88) | (−9.28/+9.62) | (−6.88/+11.89) |
| Karnofsky | +3.75 ± 11.88 | +5.38 ± 10.50*** | −2.94 ± 17.23 | +0.56 ± 16.62 | −2.16 ± 11.28 | +2.50 ± 11.98 |
| index (units) | CI: (−6.18/+13.68) | (−0.96/+11.73) | (−11.80/+5.92) | (−7.71/+8.82) | (−7.96/+3.65) | (−3.46/+8.46) |

*data show the means ±S.D. and 95% confidence intervals (C.I.) of the measurements at the 6 mths visit
^{†}measurements at the end of the observation period from patients completing at least two visits
^{‡}Means of 3 values for each subject i.e., the measurements at 6 wks, 3 mths and 6 mths
Bold data indicate a statistical significance
^{§}P = 0.049 for difference between groups
^{||}P = 0.036 for difference between groups
**P = 0.015 for difference to baseline
^{††}P = 0.010 for difference between groups, by Kruskal-Wallis test
^{‡‡}P = 0.022 for difference to baseline
^{§§}P = 0.005 for difference between groups, by Kruskal-Wallis test
^{|| ||}P = 0.019 for difference to baseline
***P = 0.089 for difference to baseline

In view of the poor condition of the patients and the resulting loss due to death or discontinuation, it seemed appropriate to analyze the data of all 35 “evaluable patients.” This analysis showed that the mean changes in body weight of the casein-treated and cysteine-rich protein-treated groups were significantly different already after 6 weeks of treatment (−1.21±3.90% (n=17) and +1.36±2.94% (n=18), respectively; p=0.038). To illustrate the changes throughout the study period, the changes between baseline and last observation according to the “last-observation-carried forward” (LOCF) method (see Appendix 8) and the means of the changes that were seen at 6 weeks, 3 months, and 6 months (or last observation) are shown in the middle and right panels of Table 3, respectively. The changes in body weight between treatment groups remained significantly different during the entire observation period (Table 3). The change in body cell mass in the cysteine-rich protein group was also different if compared either with the control group (p=0.01) or with the baseline value (p=0.02). However, the body cell mass data are less precise, as indicated by the large standard deviation, and may therefore be viewed only as supportive evidence.

The two treatment arms showed no significant differences in the changes in laboratory end points, including C-reactive protein, TNF-α, IL-6, and albumin concentrations (data not shown). The changes in glutathione (−58.9±242.7 vs. ±110.7±300.7 μM) (see Appendix 7) and plasma cysteine (+9.8±23.5 vs. ±34.8:': 150A}−LM) showed higher values for patients treated with the cysteine-rich protein but were not significantly different. The high standard deviations indicate that these data are not very precise.

### III. Effects on Changes in Quality of Life (See Appendix 6)

Several quality-of-life parameters, including hand-grip force, Karnofsky status, the feeling of being nervous and worried (McGill QOLC2), and appetite and depression (ESAS) (see Appendix 6) showed a marked deterioration shortly before death, as indicated by the data from a subgroup of six patients in whom these measurements had been performed within the last 17 days before death. These six patients showed a significantly (p<0.05) greater decrease in Karnofsky index and three other quality-of-life parameters (feeling nervous and worried, not feeling well, and depression) than the other 29 patients, irrespective of the treatment group. In the remaining 29 patients (who composed the majority of the population and included two patients whose last data had been determined 41 and 56 days before death, respectively), the group with the cysteine-rich protein showed a significant improvement in all of these parameters if compared with baseline values, whereas the casein-treated group showed no significant improvement (Table 4). Also, no significant changes were seen in other McGill or ESAS parameters (data not shown). The combined treatment arms of the imminently dying patients and the combined arms of the remaining patients were significantly different in the changes in Karnofsky status, feeling nervous and worried, not feeling well, and degree of depression (not shown). The cut-off point between day 17 and day 41 has been arbitrarily chosen.

TABLE 4
Changes based on last measurements
determined >17 days before death

| Rel. or abs. changes: | Casein | CysP |
|---|---|---|
| n | 13 | 16 |
| Body weight (%) | −3.09 ± 8.60^{§} | +2.46 ± 6.90^{§} |
| Hand Grip force(%) | 1.71 ± 21.45 | +9.10 ± 16.62^{∥} |
| | | CI: +0.24/+17.96 |
| Karnofsky index*** (units) | +2.31 ± 13.01 | +5.00 ± 9.66** |
| McGill QOLC2(units)*** | 0.23 ± 3.24 | −1.53 ± 2.50^{††,‡} |
| Feeling nervous & | | CI: 2.92/−0.15 |
| worried | | |
| ESAS 2 (units)*** | −0.85 ± 3.83 | −1.20 ± 2.68^{‡‡,‡} |
| Not feeling well* | | |
| ESAS 5 (units) | −1.23 ± 5.40 | −2.07 ± 2.60^{§§,‡} |
| Lack of Appetite | | CI: (−3.51/−0.62) |
| ESAS 11 (units)*** | −0.54 ± 4.22 | −1.60 ± 2.77^{∥∥,‡} |
| Depression | | CI: −3.14/−0.06 |

CysP = cysteine-rich protein
*Subjective feeling of physical, emotional, social, spiritual and financial well-being
^{‡}McGill QOL and ESAS data available from 15 patients only
For detailed information about McGill QOL and ESAS parameters see Ref 23-25.
Bold data indicate a statistical significance.
^{§}P = 0.064 for difference between groups
^{∥}P = 0.044 for difference to baseline
**P = 0.056 for difference to baseline
^{††}P = 0.033 for difference to baseline
^{‡‡}P = 0.105 for difference to baseline
^{§§}P = 0.008 for difference to baseline
^{∥∥}P = 0.042 for difference to baseline

### IV. Clinical Assessment of Disease Activity

The reported changes in disease activity (regression, stable disease, or progression) in the two treatment groups were not significantly different.

### V. Adverse Reactions

Of the 33 lung cancer patients allocated to the cysteine-rich protein, four complained about events possibly related to the protein (i.e., one person with moderate nausea, one with moderate nausea plus abdominal discomfort, one with mild emesis, and one with mild dryness of oral mucous membrane. One patient complained of mild increased nausea that was considered probably related to the protein. Among the colorectal cancer patients allocated to the cysteine-rich protein, one patient reported serious vomiting, moderate diarrhea, and moderate nausea, which were altogether considered probably related to the protein. Among the 33 lung cancer patients allocated to casein, one patient reported a mild case of nausea probably related to the protein, and three patients reported events possibly related to the protein (i.e., a case of mild transient vomiting, and one case each of mild and moderate constipation). Of the colorectal cancer patients allocated to casein, one reported mild nausea, which was considered definitely related to the protein, and one reported mild constipation that was possibly related to the protein.

The results show that the survival of non-small cell lung cancer patients with chemotherapy or radiotherapy or both was not decreased by supplementation with the cysteine-rich protein. Indeed, the results show that patient survival improved by supplementation with the cysteine-rich protein derived from undenatured whey. The results thus not only alleviate the concern that treatment with certain antioxidants may interfere with the tumor-cytotoxic effects of chemotherapy and radiotherapy and thus increase mortality (see 4, 9, 24). They also alleviate the concern that certain nutritional programs may enhance tumor growth.

The data, including consideration of patient compliance and statistical analysis show that treatment with approximately 5 to 30 g/day of the cysteine-rich protein is sufficient to increase patient survival in addition to reversing cancer-related weight loss and the loss of body cell mass in non-small cell lung cancer patients. Although average patient consumption was about 13 g/day, based on the amounts consumed, the amount administered should be in the range of about 5 to 30 g/day, preferably about 5 to 25 g/day, more preferably about 13-20 g/day and most preferably about 13 g/day to accomplish improvement in cancer patient survival receiving chemotherapy and/or radiation therapy. This was coincidentally associated with an improvement of muscle force and certain quality-of-life parameters, provided that measurements shortly before death were excluded. As glutathione and cysteine measurements did not yield significant differences, the trial produced no data proving the trial hypothesis that the reversal of weight loss may be largely mediated by cysteine and glutathione. The available data do not exclude that amino acids other than cysteine or other properties of the cysteine-rich protein such as digestibility may (also) contribute to the observed efficacy. However, the important role of cysteine was supported by the facts that increases in body cell mass have previously been observed in studies with other cysteine derivatives (16,20) and that the two proteins in the present study differed most strongly in cysteine content. The whey-derived protein was especially designed to have an even higher cysteine content than normal whey protein, and cysteine is a biosynthetic precursor not only of proteins but also of the cellular antioxidant, glutathione. The facts that the decrease in body cell mass in old age and cancer patients was found to be correlated with an oxidative shift in the plasma redox status (16), and certain redoxsensitive signalling pathways are involved in catabolic processes (reviewed in II), also support a role of glutathione in wasting. This paradigm links the differential effects of the two proteins in our trial to established molecular mechanisms. The trial confirmed an important prediction of the hypothesis. Oxidative stress has also been implicated in the adverse effects of chemotherapy and/or radiotherapy (4, 9, 19, 23). Postradiotherapy plasma glutathione was associated with outcome in patients with head and neck squamous cell carcinoma (3). Nevertheless, the data clearly show that supplementing patients with a cysteine-rich undenatured whey derived protein did not interfere with the tumor-cytotoxic effects of chemotherapy and/or radiotherapy to increase mortality; and in fact resulted in an increase in patient survival.

The superior efficacy of the cysteine-rich protein in comparison to casein is important for these patients, in view of their early satiety and the resulting inability to eat more protein. Both treatment groups showed a mean compliance of <50% corresponding to about 13 g protein/day. This was reminiscent of the generally low dietary protein intake in cancer patients (14, 21).

The study also demonstrates the efficacy of the cysteine-rich protein to support weight gain and increase in body cell mass in patients with advanced non-small cell lung cancer. Muscle function and several quality-of-life parameters were also improved, except in imminently dying patients. The data suggest that the potential efficacy of a therapeutic intervention may be missed if data from imminently dying patients are mixed with data from patients surviving all or a large part of the study period.

### VI. Abbreviations

CRP, C-reactive protein; EPO, erythropoietin; ESAS, Edmonton symptom Assessment Scale; HR, hazard ratios; LOCF, last observation carried forward; PP, per protocol; QOL, quality of life; TNF-a: tumor necrosis factor a.

### VII. Appendix

### Patients and Methods

### 1. Study Design

This multicenter, randomized, double-blind phase II study (Health Canada Number 085608) was designed to evaluate the safety and efficacy of the cysteine-rich protein isolate, IMN 1207 (Immunotec Research Ltd., Vaudreuil, QC, Canada) in the prevention of wasting in two strata of patients (i.e., patients with metastatic colorectal and stage IIIB-IV non-small cell lung cancer, respectively, over 6 months). The study treatment consisted of oral administration of the cysteine-rich protein or another protein, casein, instead of a placebo. Participants received seven 400-g canisters of the powdered medications twice (i.e., at the start and at the 3-month visit. They also received a 10-g scoop, shaker, blender, and instructions to ingest three scoops (3×1O g) daily.

The study medication was administered in conjunction with standard of care for cancer type and stage.

### 2. Study Medication

The amino acid composition of the cysteine-rich protein and the control protein (Table 1) was determined by using an amino acid analyzer.

Preparation of Cysteine-Rich Protein and Casein, Labelling, Packaging, Stability testing, and shipment were conducted by Immunotec Research, Ltd., in collaboration with Well Spring Pharmaceutical Corporation, Canada. The products met all stability testing specifications of Health Canada.

### 3. Inclusion and Exclusion Criteria

Included were metastatic colorectal or non-small-cell lung cancer patients in two separate strata. Here we report the results from the lung cancer patients only. Included were patients of 21 years or older with an involuntary decrease in body weight of >3% during 3 months immediately preceding study entry, Karnofsky performance status; 0:70%, life expectancy >3 months, serum creatinine <3.0 mg/dl or 265/LM, bilirubin in the normal range, and SGPT/ALT <6 times the upper limit of normal and reliable contraception in the case of women of childbearing potential. Excluded were patients with a history of angioedema, allergic reactions to any agent used in this study, uncontrolled metastatic brain tumors, milk protein intolerance, ascites, edema, significant anemia, or subjects currently using N-acetylcysteine, alpha-lipoic acid, or dry whey protein supplements.

### 4. Determination of Sample Size

Based on historic data, a mean decrease of 4% body weight over a 3-month period with a standard deviation of 4% was expected in the control group. With p=0.05, a sample size of 2×30 patients was expected to give an 80% chance of detecting a hypothetical 3% difference in weight loss between treatment groups.

### 5. Recruitment, Randomization, and Evaluation

Patient randomization and data management were performed by GEREQ, Montreal, Quebec. Data monitoring and site visits were performed by Canreg, Inc., Dundas, Ontario. Patients were recruited and screened for eligibility at the clinical trial centers (i.e., Department of Oncology at McGill University, Montreal; Cross Cancer Institute, Edmonton; Juravinski Cancer Center, Hamilton; and Allan Blair Cancer Center, Regina). At a central independent biostatistical company (GEREQ), patients were subsequently stratified into two strata with lung cancer and colorectal cancer, respectively, and randomized into the two treatment arms. The blinding code was kept at the statistician's office. At the trial sites, randomized patients underwent clinical examinations before the start of treatment (week 0), and at week 6, month 3, and month 6. At these time points, additional blood and urine samples were taken for laboratory tests, the Karnofsky performance status, quality of life (QOL), symptoms, and appetite (8), as well as ESAS (6, 7), and hand-grip strength were determined, and a CT scan or MRI was performed as part of patient's cancer therapy. Laboratory tests included hematologic profile, clinical biochemistry, biologic markers, urine analysis, and pregnancy tests (women only). The data were recorded at the trial sites. Compliance, as defined by the intake of study medication, was determined by weighing the returned canisters. Safety was continuously monitored by central collection of records of all serious adverse events including mortality.

### 6. Primary and Secondary End Points

Primary end points were the relative (%) change in body weight and percentage change in absolute body cell mass over a 6-month period.

Secondary end points included the assessment of strength by hand-grip dynamometry. Karnofsky performance status. assessment of the quality of life based on the McGill QOL (8), and “symptom burden” based on the Edmonton Symptom Assessment Scale (ESAS) (6, 7), mortality, changes in glutathione in red blood cells, plasma concentrations of cysteine, erythropoietin (EPO), interleukin 6 (IL-6), tumor necrosis factor alpha (TNF-α), and C-reactive protein (CRP), and disease activity (regression, stable disease, or progression).

### 7. Determination of Body Cell Mass and Blood Parameters

Body cell mass (16) was determined by bioelectrical impedance analysis [Biodynamics (Model 450), Seattle, Wash.]. Patients were advised to drink only 400 ml and not to eat during the last 8 hours before analysis. Clinical assessments and some laboratory tests including C-reactive protein (CRP) were performed directly at the trial sites. Plasma amino acids, EPO, IL-6, and TNF-alpha were analyzed centrally by the Clinical Research and Clinical Trials Laboratory, Hamilton, Ontario, Canada. Free plasma amino acids including cysteine were determined by using an amino acid analyzer. Glutathione was assayed by Immunosciences Lab., Inc., Beverly Hills, Calif., U.S.A., by using the commercial test kit BIOXYTECH R GSH-420™ (catalog no. 21023). Redblood cells were obtained from 0.5 ml blood by centrifugation for 5 min at 2,500 g and 4° C. After removal of the plasma, the cells were washed 3 times in cold saline, resuspended in 4 volumes of cold water, and vortexed thoroughly. A volume of 0.1 ml lysate was then mixed with 0.3 ml of “precipitation reagent” (aqueous solution of trichloroacetic acid) in a microcentrifuge tube, vortexed for at least 15 sec, and then centrifuged at 10,000 g for 5 min at room temperature.

A volume of 0.2 ml of the resulting supernatant was thoroughly mixed with 0.2 ml of a pH 7.8 “buffer” (potassium phosphate/diethylene triaminepentaacetic acid lubrol) and 0.2 ml “reducing agent” [tris(2-carboxyethyl)phosphine in HCl]. After addition of 0.2 ml “chromogen” (l-methyl-4-chloro-7-trifluoromethylquinolinium methylsulfate in HCl), the solution was again thoroughly mixed and subsequently mixed with 0.2 ml “color developer” (aqueous NaOH solution). After incubation for 30 min at room temperature in the dark, absorbance was measured at 420 nm.

### 8. Statistical Analysis

The statistical analysis was performed by an independent statistical company (Boreal Primum, Montreal, Quebec). The data were analyzed separately for the two strata (disease sites) and expressed as means±SD. Treatment effects were statistically evaluated by comparison with the control group (two-sided t test for independent variables, if not indicated otherwise) and by comparison with the corresponding baseline values (t test for dependent variables). Comparison of the treatment groups was based on the data obtained at 6 months or on the last-observation-carried-forward (LOCF) method for patients who completed at least two visits. Survival curves were generated with the use of Kaplan-Meier estimates for treatment and compared with the log-rank test. Hazard ratios (HRs) were estimated by using a Cox proportional-hazard model. The results were judged by the p value. A p value <0.05 was regarded as statistically significant.

Although specific preferred embodiments of the invention have been described above with reference to the company figures, it will be apparent to a skilled person that the invention is not limited to those precise embodiments and that many modifications and variations could be effected therein by one skilled in the art without departing from the spirit or scope of the invention as defined in the appended claims.

### REFERENCES

Reference is made to certain articles listed below, the contents of which are incorporated by reference herein. These publications are referred to by numerals within parenthesis appearing in this specification.
1. Barber M D. Ross J A. Voss A C. Tisdale M J. and Fearon K C. The effect of an oral nutritional supplement enriched with fish oi] on weight-]oss in patients with pancreatic cancer. Br J Cancer 8]: 80-86. 1999.
2. Bauer J D. Capra S. Nutrition intervention improves outcomes in patients with cancer cachexia receiving chemotherapy: a pilot study. Support Care Cancer 13: 270-274. 2005.
3. Bohn S K. Smeland S. Sakhi A K. Thoresen M. Russnes K M. Tausso J. Svilaas A. Svilaas T. and Blomhoff R. Post-radiotherapy plasma total glutathione is associated to outcome in patients with head and neck squamous cell carcinoma. Cancer Lett 238: 240-247. 2006.
4. Borek C. Dietary antioxidants and human cancer. Integr Cancer Ther 3: 333-341. 2004
5. Bruera E. ABC of palliative care: anorexia, cachexia and nutrition. BMJ 315: 1219-1222. 1997.
6. Bruera E. Kuehn N. Miller M J. The Edmonton Symptom Assessment System (ESAS): a simple method for the assessment of palliative care patients, J Palliat Care 7: 6-9, 1991.
7. Chang V T. Hwang S S. Feuerman M. Validation of the Edmonton Symptom Assessment Scale. Cancer 88: 2164-2171. 2000.
8. Cohen S R. Mount B M. Living with cancer: “good” days and “bad” days: what produces them? Can the McGill Quality of Life Questionnaire distinguish between them? Cancer 89: 1854-1865. 2000.
9. Coia L R. Moyland D J. Introduction to clinical radiation oncology. Madison. Wis.: Medical Physics Publishing, 1998.
10. Davidson W, Ash S, Capra S, Bauer J, and Cancer Cachexia Study Group. Weight stabilization is associated with improved survival duration and quality of life in unrespectable pancreatic cancer. Clin Nutr 23: 239-247, 2004.
11. Orage W. Redox regulation in anabolic and catabolic processes. Curr Opin Clin Nutr Metab Care 9:]90-195. 2006.
12. Evans W K, Nixon D V, Daly J M. A randomized trial of oral nutritional support versus ad lib nutritional intake during chemotherapy for advanced colorectal and non-small-cell lung cancer. J Clin Oncol 5: 113-124, 1987.
13. Fearon K C, Voss A C, and Hustead D S. Definition of cancer cachexia: effect of weight loss, reduced food intake, and systemic inflammation on functional status and prognosis. Am J Clin Nutr 83: 1345-1350, 2006.
14. Fearon K C H, von Meyenfeldt M F, Moses A G W, Van Geenen R, Roy A, Gouma D I, Giacosa A, Van Gossum A, Bauer J, Barber M D, Aaronson N K, Voss A C, and Tisdale M I. Effect of a protein and energy dense n-3 fatty acid enriched oral supplement on loss of weight and lean tissue in cancer cachexia: a randomized double blind trial. Gut 52: 1479-1486, 2003.
15. Glare P. Clinical predictors of survival in advanced cancer. J Support Oncal 3: 331-339, 2005.
16. Hack V, Breitkreutz R, Kinscherf R, Rohrer H, Biirtsch P, Taut F, Benner A, and Droge W. The redox state as a correlate of senescence and wasting and as a target for therapeutic intervention. Blood 92: 59-67, 1998.
17. Hutton J L, Martin L, Field C J, Wismer W V, Bruera E D, Watanabe S M, and Bracos V E. Dietary patterns in patients with advanced cancer: implications for anorexia-cachexia therapy. Am J Clin Nutr 84: 1163-1170, 1998.
18. Inagaki J, Rodriguez V, Bodey G P. Causes of death in cancer patients. Cancer 33: 568-573, 1974.
19. Jonas C R, Puckett A B, Jones D P, Griffith D P, Szeszycki E E, Bergman G F, Furr C E, Tyre C, Carlson J L, Galloway J R, Blumberg 18, and Ziegler T R. Plasma antioxidant status after high-dose chemotherapy: a randomized trial of parenteral nutrition in bone marrow transplantation patients. Am J Clin Nutr 72: 181-189, 2000.
20. Mantovani G, Madeddu C, Maccio A. Cancer-related anorexia/cachexia syndrome and oxidative stress: an innovative approach beyond Current treatment. Cancer Epidemial Biomarkers Prevent 13: 1651-1659, 2004.
21. Millward D J, Jackson A A. Protein/energy ratios of current diets in developed and developing countries compared with a safe protein/energy ratio: implications for recommended protein and amino acid intakes. Public Health Nutr 7: 387-405, 2004. [Review].
22. Ovesen L, Allingstrup L, Hannibal J, Mortensen E L, and Hansen O P. Effect of dietary counseling on food intake, body weight, response rate, survival, and quality of life in cancer patients undergoing chemotherapy: a prospective, randomized study. J Clin Oncol 11: 2043-2049, 1993.
23. Schmidt-Ullrich R K. Molecular targets in radiation oncology. Oncogene 22: 5730-5733, 2003.
24. Seifried H E, Anderson D E, Sorkin B C, and Costello R B. Free radicals: the pros and cons of antioxidants. J Nutr 134: 3143S-3163S, 2004.
25. Stratton R J, Elia M. A critical, systematic analysis of the use of oral nutritional supplements in the community. Clin Nutr 18: S29-S84, J 999.
26. Warren S. The immediate causes of death in cancer. Am J Med Sei 184: 610-615, 1932.
27. Yavuzsen T, Davis M P, Walsh D, LeGrand S, and Lagman R. Systematic review of the treatment of cancer-associated anorexia and weight loss. J Clin Oncol 23: 8500-851 J, 2005.

1. A method of increasing survival of a patient, said method comprising administering a cysteine-rich, undenatured whey-derived protein to a patient, wherein the patient is undergoing chemotherapy, radiation therapy, or both.

2. The method of claim 1, wherein the protein is administered to the patient in an amount of about 5 to 30 g/day.

3. The method of claim 1, wherein the patient is being treated for cancer.

4. The method of claim 2, wherein the patient is being treated for cancer.

5. The method of claim 1, wherein the patient is being treated for lung cancer.

6. The method of claim 2, wherein the patient is being treated for lung cancer.

7. The method of claim 1, wherein the cysteine-rich, undenatured whey-derived protein comprises 0.5 moles cysteine per/kg.

8. The method of claim 2, wherein the cysteine-rich, undenatured whey-derived protein comprises 0.5 moles cysteine per/kg.

9. The method of claim 1, wherein the protein is administered to the patient in an amount of about 5 to 25 g/day.

10. The method of claim 1, wherein the protein is administered to the patient in an amount of about 13 to 20 g/day.

11. The method of claim 1, wherein the protein is administered to the patient in an amount of about 13 g/day.

12. The method of claim 1, wherein the patient is further improved in one or more of cancer-related weight loss, body cell mass, hand grip force, quality of life, Karnofsky status, feelings of anxiety, feelings of nervousness, feelings of worry, appetite, and depression.

13. The method of claim 2, wherein the patient is further improved in one or more of cancer-related weight loss, body cell mass, hand grip force, quality of life, Karnofsky status, feelings of anxiety, feelings of nervousness, feelings of worry, appetite, and depression.

14. The method of claim 3, wherein the patient is further improved in one or more of cancer-related weight loss, body cell mass, hand grip force, quality of life, Karnofsky status, feelings of anxiety, feelings of nervousness, feelings of worry, appetite, and depression.

15. The method of claim 4, wherein the patient is further improved in one or more of cancer-related weight loss, body cell mass, hand grip force, quality of life, Karnofsky status, feelings of anxiety, feelings of nervousness, feelings of worry, appetite, and depression.
